(19) United States
(12) Patent Application Publication
(10) Pub. No.: US 2013/0110204 A1
(43) Pub. Date: May 2, 2013

(54) SPRING CONNECTOR FOR IMPLANTABLE MEDICAL DEVICE

(71) Applicants: Wisit Lim, Santa Clarita, CA (US); Xiangqun Chen, Santa Clarita, CA (US); Mitch Goodman, Saugus, CA (US)

(72) Inventors: Wisit Lim, Santa Clarita, CA (US); Xiangqun Chen, Santa Clarita, CA (US); Mitch Goodman, Saugus, CA (US)

(73) Assignee: PACESETTER, INC., Sylmar, CA (US)

(21) Appl. No.: 13/282,218

(22) Filed: Oct. 26, 2011

(51) Int. Cl. A61N 1/36

(52) U.S. Cl. 607/72

(57) ABSTRACT

Implementations of the present disclosure involve an implantable medical pulse generator for administering electrotherapy via an implantable medical lead having a lead connector end on a proximal end of the lead. The pulse generator may include a can and a header coupled to the can. The header may include a lead connector end receiving receptacle for transmitting electrical pulses from the can to the lead through one or more electrical spring contacts in electrical communication with one or more terminals of the lead connector end. The one or more spring contacts may include a triangular shaped metal spring within a housing that forms an electrical contact with the lead connector end when the one or more terminals of the lead connector end is inserted into the header. Alternatively, the metal spring may take other shapes, such as, for example, circular, elliptical, or rectangular.

### CROSS REFERENCE TO RELATED APPLICATIONS

The present application is related to copending U.S. patent application Ser. No. ______, filed ______, titled “SPRING CONNECTOR FOR IMPLANTABLE MEDICAL DEVICE” (Atty. Docket A11P1037US01), which application is incorporated by reference in its entirety into the present application.

### FIELD OF THE INVENTION

Aspects of the present invention relate to medical apparatus and methods. More specifically, the present invention relates to apparatus for electrically connecting an implantable medical lead to an implantable pulse generator.

### BACKGROUND OF THE INVENTION

An implantable pulse generator such as an artificial pacemaker or implantable cardioverter defibrillator (“ICD”) is a medical device which uses electrical impulses to regulate the beating of a heart. In general, the pulse generator administers electrical impulses to the appropriate heart tissue vie one or more leads inserted transvenously with distal ends of the leads located within the chamber or chambers of the heart. The distal ends of the leads support electrodes for sensing, pacing and defibrillation.

After placement of the electrodes, the proximal ends of the leads may be physically and electrically connected to the pulse generator. In some instances, the pulse generator may be placed below the subcutaneous layer of the chest. The pulse generator utilizes a battery or other power source to generate the electrical impulses which are transmitted through conductors in the leads to the electrodes at the lead distal end and into the heart for regular pacing. As such, the electrical connection between the proximal ends of the leads and the pulse generator is of significant importance to provide a steady and regular pace to the heart.

### BRIEF SUMMARY OF THE INVENTION

One implementation of the present disclosure may take the form of an implantable medical pulse generator for administering electrotherapy via an implantable medical lead having a lead connector end on a proximal end of the lead. The pulse generator may include a can and a header coupled to the can. The header may include a lead connector end receiving receptacle and a conductive spring contact configured to be in electrical contact with a terminal of the lead connector end when the lead connector end is inserted into the receiving receptacle. In addition, the conductive spring contact may include a cylindrically shaped housing including a bore through the body of the housing, a triangularly shaped metal spring located at least partially within the bore of the housing and oriented such that at least an inner portion of the spring protrudes into the bore of the housing.

Another implementation of the present disclosure may take the form of an implantable medical pulse generator. The pulse generator may comprise a can and a header coupled to the can. The header may include a lead connector end receiving receptacle and a spring contact associated with the lead connector end receiving receptacle. Additionally, the conductive spring contact may include a cylindrically shaped housing including a bore through the body of the housing, a non-circular shaped metal spring located at least partially within the bore of the housing and oriented such that at least an inner portion of the spring protrudes into the bore of the housing.

Yet another implementation of the present disclosure may take the form of an apparatus for administering electrotherapy via an implantable medical lead having a lead connector end on a proximal end of the lead. The apparatus may include a can configured to generate an electrical pulse and a connector assembly configured to transmit the generated electrical pulse to the lead connector end of the medical lead through a conductive spring contact in electrical communication with a terminal of the lead connector end. Additionally, the conductive spring contact may include a cylindrically shaped housing including a bore through the body of the housing of at least a first circumference and a groove located within the bore of the housing, the groove having a circumference larger than the first circumference. Further, the conductive spring contact may also include a triangularly shaped metal spring located at least partially within the groove and oriented such that the corners of the triangularly shaped metal spring are at least partially recessed within the groove.

Another implementation of the present disclosure may take the form of a pulse generator for administering electrotherapy via an implantable medical lead including a lead connector end on a proximal end of the lead, the lead connector end including a terminal. The pulse generator includes a lead connector end receiving receptacle having a bore with a longitudinal axis and a conductive spring contact having a spring with multiple coils helically extending about a longitudinal axis of the spring. The longitudinal axis of the spring is generally coaxially aligned with the longitudinal axis of the bore. At least one of the coils has a non-circular shape when viewed along the longitudinal axis of the spring.

Yet another implementation of the present disclosure may take the form of a method of manufacturing a spring contact of an implantable medical pulse generator. The method begins by providing a continuously extending wire. The continuously extending wire is then helically wound into a helix having a continuous wire. In helically winding the wire, the wire is formed into multiple coils of the helix, the coils being formed to have a non-circular shape when viewed along a longitudinal axis of the helix. A helical spring is then cut to a desired length from the helix. The helical spring is then positioned in an electrically conductive ring of the spring contact so a longitudinal axis of the spring is generally coaxially aligned with a longitudinal axis of the ring.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1 is an isometric view of a proximal end portion (i.e., lead connector end) of a conventional transvenous bipolar pacing lead.

FIG. 2 is an isometric view of a cardiac pacemaker/defibrillator unit (i.e., pulse generator) incorporating connector junctions for communication with one or more electrodes.

FIG. 3 depicts an enlarged isometric view of the connector assembly of the pulse generator of FIG. 2.

FIG. 4 is a lateral cross-sectional view of the connector assembly as taken along section line 4-4 of FIG. 3.

FIG. 5 is an isometric view of a spring contact of the pulse generator depicted in FIGS. 3 and 4.

FIG. 6 is an isometric view of the housing of the spring contact of FIG. 5.

FIG. 7 is a longitudinal cross-sectional view of the housing of the spring contact as taken along section line 7-7 of FIG. 6.

FIG. 8 is an isometric view of a triangular shaped spring of the spring contact of FIG. 5.

FIG. 9 is an isometric view of an end cap of the spring contact of FIG. 5.

FIG. 10 is an exploded view of the assembly of the spring contact of the connector assembly, including the housing, the triangular metal spring and the end cap.

FIG. 11 is an isometric longitudinal cross-sectional view of the spring contact of the connector assembly along section line 11-11 of FIG. 5.

FIG. 12 is an isometric view of the spring contact of the connector assembly engaging a lead connector end on a proximal end of a lead.

FIG. 13 is an isometric view of an extended triangular metal spring utilized for the triangular metal spring of the spring contact of FIG. 5.

FIG. 14 is an isometric view of a second embodiment of a triangular metal spring of the spring contact of the connector assembly.

FIG. 15 is an isometric view of a third embodiment of the spring of the spring contact of the connector assembly.

FIG. 16 is an axial end elevation of the spring of FIG. 15.

FIG. 17 is a side elevation of the spring of FIG. 15.

FIG. 18 is an axial end elevation of a single loop or coil of the spring of FIGS. 15-17.

FIG. 19 is an isometric view of a fourth embodiment of the spring of the spring contact of the connector assembly.

FIG. 20 is an axial end elevation of the spring of FIG. 19.

FIG. 21 is a side elevation of the spring of FIG. 19.

FIG. 22 is a side elevation of spring of FIG. 19.

FIGS. 22A-A, 22B-B, 22C-C and 22D-D are transverse cross sections of the spring respectively taken along section lines A-A, B-B, C-C and D-D of FIG. 22.

FIG. 23 is a side elevation of spring of FIG. 19.

FIGS. 23A-A, 23B-B, 23C-C and 23D-D are transverse cross sections of the spring respectively taken along section lines A-A, B-B, C-C and D-D of FIG. 23.

FIG. 24 is an enlarged view of region A in FIG. 23B-B.

FIGS. 25A and 25B, are, respectively, an axial view and a partial longitudinal cross section of the proximal end (i.e., pin contact) of the lead connector end entering the spring contact and just beginning to make contact with the spring.

FIGS. 26A and 26B are, respectively, the same views as FIGS. 25A and 25B, except with the pin contact of the lead connector end is received within the spring of the spring contact.

### DETAILED DESCRIPTION

Implementations of the present disclosure involve an implantable medial pulse generator for administering electrotherapy via an implantable medical lead having a lead connector end on a proximal end. The pulse generator may include a can and a header coupled to the can. The header may include a first lead connector end receiving receptacle for transmitting electrical pulses from the can to the lead through one or more electrical spring contacts in electrical communication with one or more terminals of the lead connector. In one embodiment, the one or more spring contacts may include a triangular shaped metal spring within a housing that forms an electrical contact with terminals of the lead connector end when the lead connector end is inserted into the header. In other embodiments, the metal spring may take other shapes, such as circular or rectangular that form an electrical contact with the lead connector.

Before a detailed discussion of the spring contact assembly of the header is provided, a general discussion is first given regarding common features of a common lead connector end at the proximal end of an implantable medical lead followed by a general discussion of the features of an implantable medical pulse generator.

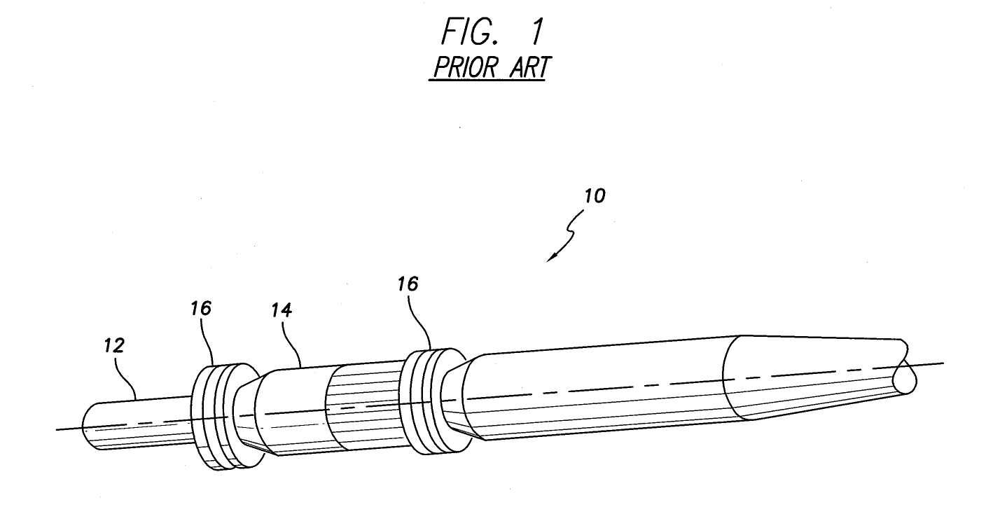

FIG. 1 shows a proximal end portion 10 of a conventional transvenous, bipolar pacing lead. The diameter of such a lead may be made a sufficiently small diameter to facilitate the lead's implantation into small veins such as those found in the coronary sinus region of the heart and to allow implantation of a plurality of leads into a single vessel for multi-site or multi-chamber pacing. It should be understood, however, that other lead designs may be used, for example, multipolar leads have proximal ends portions that are bifurcated, trifurcated or have other branched configurations. While the lead whose proximal end is shown in FIG. 1 is of the bipolar variety, there are unipolar leads that carry but a single electrode, and multipolar leads that have more than two electrodes.

As is well known in the art, bipolar coaxial leads typically consists of a tubular housing of a biocompatible, biostable insulating material containing an inner multifilar conductor coil that is surrounded by an inner insulating tube. The inner conductor coil is connected to a tip electrode on the distal end of the lead. The inner insulating tube is surrounded by a separate, outer multifilar conductor coil that is also enclosed within the tubular housing. The outer conductor coil is connected to an anodal ring electrode along the distal end portion of the lead. The inner insulation is intended to electrically isolate the two conductor coils preventing any internal electrical short circuit, while the housing protects the entire lead from the intrusion of body fluids. These insulating materials are typically either silicone rubber or polyurethane. More recently, there have been introduced bipolar leads in which multifilar cable conductors contained within multilumen housings are substituted for the conductor coils in order to reduce even further the overall diameter of the lead.

The proximal lead end portion 10 shown in FIG. 1 conforms to the IS-1 standard, comprising a pair of coaxial spaced-apart terminals including a tip terminal 12 and a ring terminal 14. The tip terminal 12 is electrically connected by means of the inner conductor coil to the tip electrode at the distal end of the lead, while the ring terminal 14 is electrically connected to the anodal ring electrode by means of the outer conductor coil. The tip and ring terminals of the lead may each be engaged by a conductive garter spring contact or other resilient electrical contact element carried by a connector assembly as described below. The proximal end portion further comprises spaced-apart pairs of seal rings 16 for preventing body fluids from reaching the electrical contacts. With the proximal end portion 10 of the lead inserted in a lead receptacle of a connector assembly, the tip and ring terminals 12 and 14 are electrically coupled via the contacts and a feedthrough to the electronic circuits within the hermetically sealed, attached cardiac pacemaker, or other implantable tissue stimulation and/or sensing device (i.e., pulse generator).

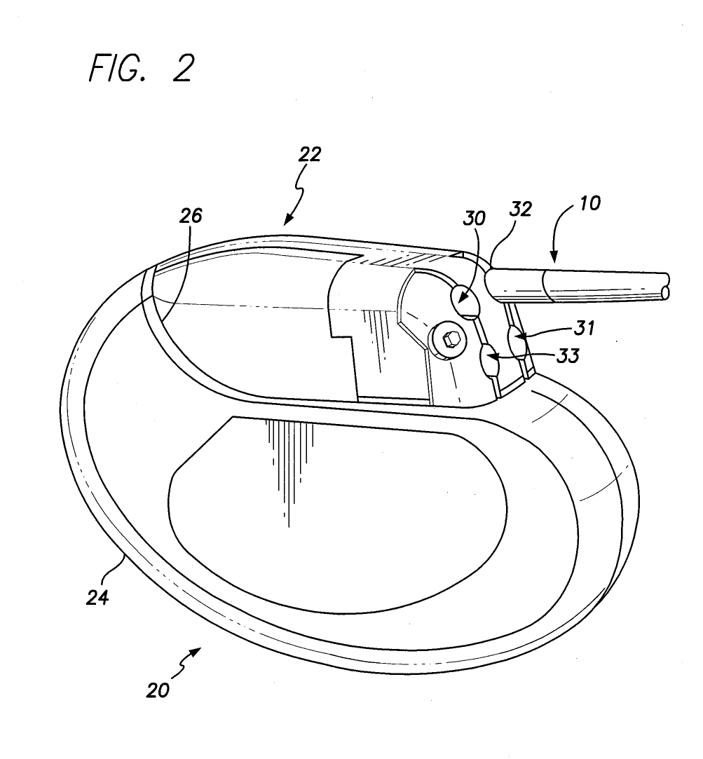

FIG. 2 shows a multi-site or multi-chamber cardiac pacemaker/defibrillator unit (i.e., pulse generator) 20 incorporating a connector assembly 22. The cardiac pacemaker/defibrillator unit 20 is of a conventional design, including a hermetically sealed can or casing 24 enclosing the electronic components of the pacemaker/defibrillator unit with the connector assembly 22 mounted along a top edge 26 of the unit.

FIG. 2 illustrates that, in some embodiments, the connector assembly 22 may include four or more receptacles 30, 31, 32 and 33 for receiving the proximal ends of four leads. FIG. 2 also shows the proximal end portion 10 of a lead inserted in a receptacle 32. In other embodiments, the connector assembly 22 includes two receptacles comprising a single pair of receptacles (i.e., receptacles 30 and 31) for receiving the proximal ends of leads such as, for example, conventional bipolar leads and/or conventional cardioverting and/or defibrillating leads.

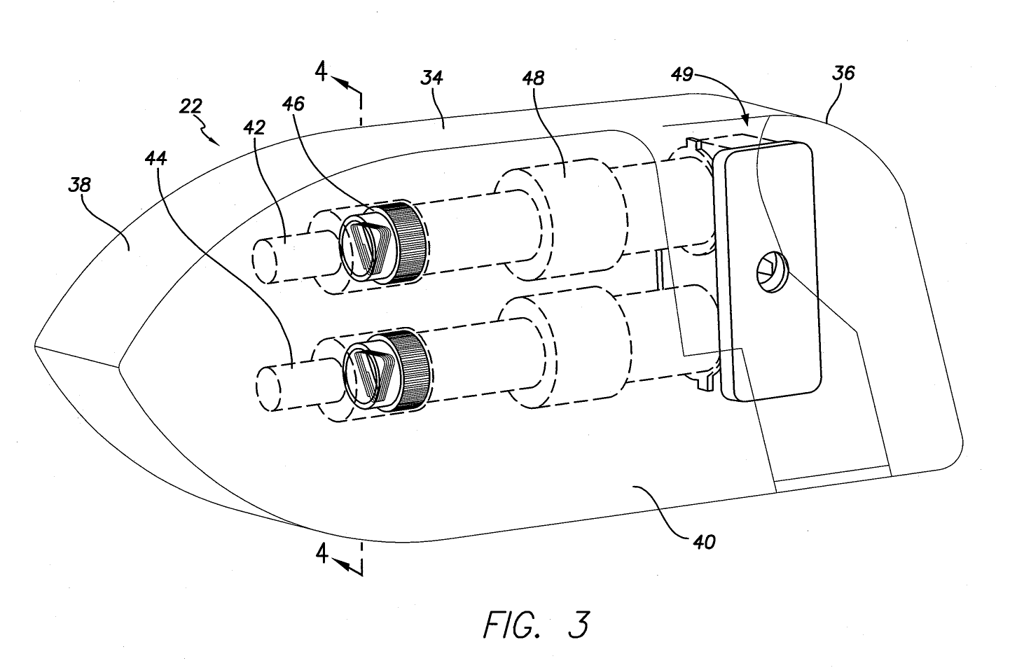

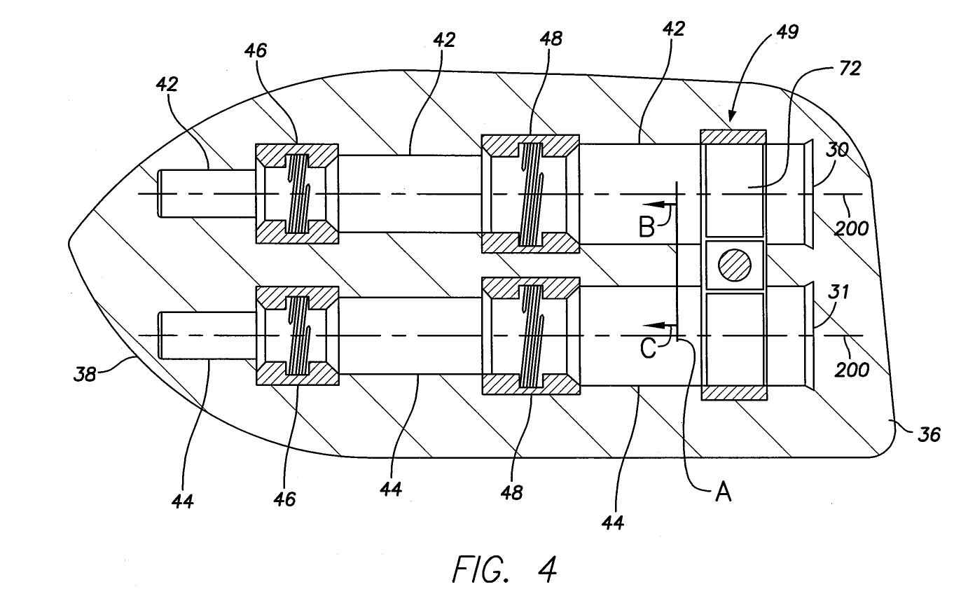

FIG. 3 depicts an isometric view of the connector assembly 22 of the pulse generator 20 of FIG. 2, except the connector assembly 22 of FIG. 3 is a version having a single pair of receptacles as opposed to two pair of receptacles. The connector assembly 22 includes a body 34 with a front or receptacle side 36, a rear side 38 and lateral sides 40 extending generally parallel to each other between the front side 36 and the rear side 38. As can be understood from FIG. 3 and FIG. 4, which is a lateral cross-sectional view of the connector assembly 22 as taken along section line 4-4 in FIG. 3, the receptacles 30 and 31 are defined in the front side 36 and extend as generally parallel bores 42 and 44 through the body 34 of the connector assembly 22. As best understood from a comparison of the bores 42 and 44 depicted in FIG. 4 and the stepped cylindrical shape of the proximal lead end portion 10 illustrated in FIG. 1, the bores have a stepped cylindrical shape that is configured to matingly receive the proximal lead portion 10.

As shown in FIGS. 3 and 4, each bore includes a most inward conductive spring contact 46 and a most outward conductive spring contact 48. As can be understood from a comparison of FIG. 4 and FIG. 1, when the proximal lead end portion 10 is received in one of the bores 42 and 44, the tip terminal 12 is received by the most inward contact 46, and the ring terminal 14 is received by the most outward contact 48. Also, the most distal seal ring 16 is located generally inward of line A, as indicated by arrows B and C, when the lead connector end 10 is fully received in the bore 42 or 44 of a receptacle 30 or 31. As a result, the most distal seal ring 16 is proximal of a lead security mechanism 49 that is used to secure the lead proximal end portion 10 in a bore 42 or 43 (see FIGS. 2-4).

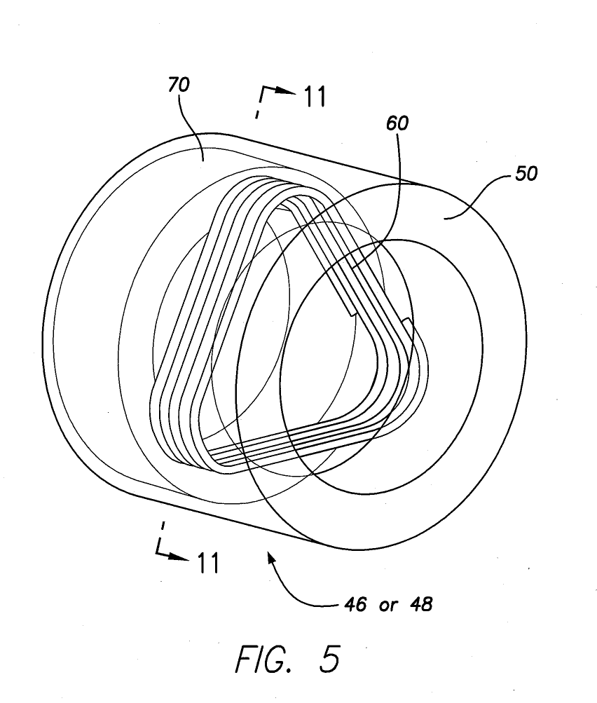

FIG. 5 is an isometric view of one of the spring contacts 46 or 48 of the connector assembly 22 depicted in FIG. 3 and FIG. 4. The spring contact 46 or 48 includes a cylindrically shaped housing 50, a triangular-shaped metal spring 60 and a cylindrical end cap 70 assembled together to form the spring contact. The components of the spring contact assembly are discussed in more detail below. As mentioned above, the spring contacts 46 or 48 electrically engage with the terminals 14 and 12 of the proximal lead end portion 10 to provide the generated electrical pulses from the can 24 of the pulse generator to the leads. In this manner, the electrical pulses are transmitted to the terminals of the proximal lead end 10 for transmission through the lead for delivery into the body via the electrode on the distal end of the lead.

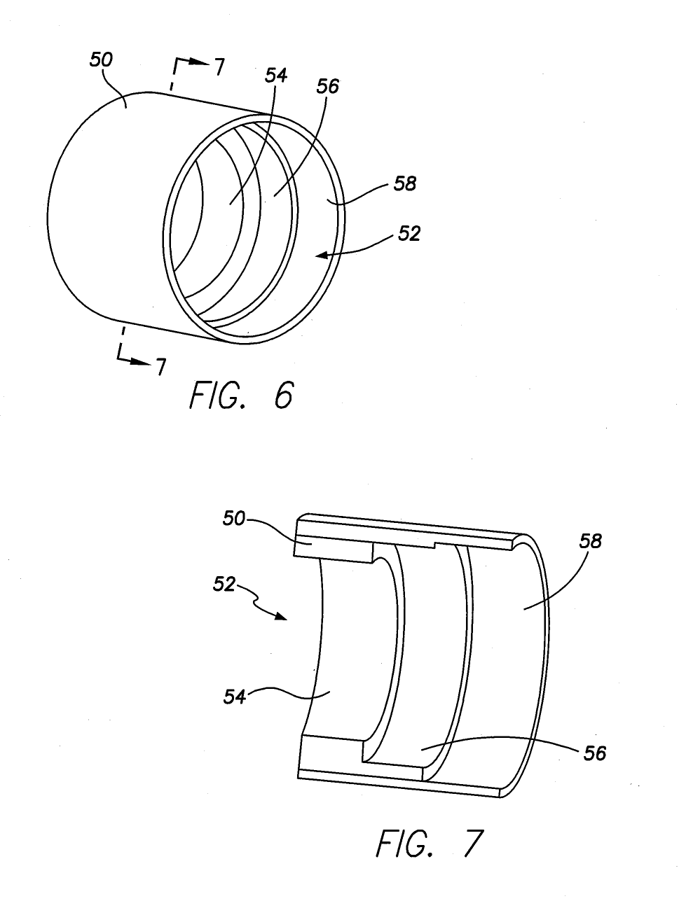

FIG. 6. shows an isometric view of the housing 50 of the spring contact 46 or 48 of FIG. 5 while FIG. 7 shows a longitudinal cross-sectional view of the housing of the spring contact as taken along section line 7-7 of FIG. 6. As can be seen in both FIGS. 6 and 7, the housing 50 includes a stepped bore 52 that defines a first inner circumferential surface 54, a second inner circumferential surface 56 with a larger circumferential diameter than the first inner circumferential surface and a third inner circumferential surface 58 with a larger circumferential diameter than the second inner circumferential surface. As explained in more detail below, the second inner circumferential surface 56 of the housing 50 may partially define a groove oriented generally perpendicular to a center axis of the bore 52. Further, the third inner circumferential surface is generally shaped to accept an end cap, described in more detail below with reference to FIG. 9, that is inserted into the housing 50 during assembly of the spring contact. In one embodiment, the housing 50 is constructed of stainless steel, titanium, MP35N or other electrically conductive materials.

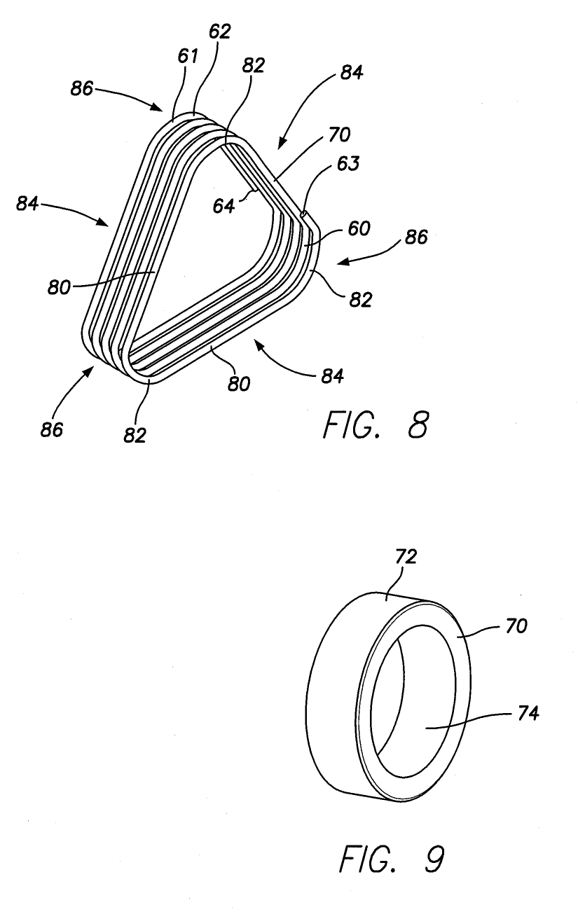

FIG. 8 is an isometric view of a triangular shaped spring 60 of the spring contact of FIG. 5. In the embodiment shown, the spring 60 has a generally triangular shape. In other words, while the embodiment depicted in FIG. 8 represents a continuously extending wire 61 helically wound into a series of coils 62 between a first free end 63 and a second free end 64 of the spring 60, the spring 60 has a triangular shape as viewed axially or, in other words, the spring 60 has a triangularly shaped cross section taken transverse to the longitudinal axis of the spring 60. The triangular shaped cross section of the helically wound spring 60 is on account of the individual coils 62 of the spring 60 having a triangular shape.

While the spring 60 may be a triangular shaped helically wound spring 60 with triangular shaped coils 62, in other embodiments, the spring 60 may be formed into other shapes, such as rectangular, pentagonal, hexagonal, octagonal, oval or circular, depending on whether the transverse cross section of the spring and the shape of its coils 62 are rectangular, pentagonal, hexagonal, octagonal, oval or circular. The spring 60 is generally formed of a circular wire 61 of stainless steel, MP35N, platinum-iridium alloy, or other electrically conductive material. To form the shape of the spring coil, a circular wire may be wrapped or wound around a mandrel of the desired shape, such as a triangular or rectangular mandrel, to create one or several loops of the wire that take the general shape of the mandrel. In other embodiments, the shape of the spring coil may be formed via CNC wire forming.

In addition, while the embodiment shown includes five to six loops or coils 62 of the circular wire, the spring 60 may have any number of loops or coils 62. For example, the spring 60 may include a single loop or several loops with any distance between the loops, as desired for creating an electrical contact point to the terminals of the lead connector of FIG. 1. Further, the circumference of the spring 60 may be similar to the dimensions of the second circumferential surface 56 of the housing 50 of FIGS. 6 and 7 such that the spring may be inserted within the housing, as shown best in FIG. 11 below.

As can be understood from FIG. 8, where the spring 60 is formed of non-circular or non-oval loops or coils 62 so as to have a corresponding non-circular or non-oval transverse cross section, each coil 62 will have linear straight sides or wire segments 80 and corner or lobes 82. Specifically, two linear straight sides 80 will intersect to form a corner or lobe 82 that may have an arcuate or curved radius. Thus, the spring 60 will have straight sides 84 intersecting at curved corners or lobes 86.

FIG. 9 is an isometric view of an end cap 70 of the spring contact of FIG. 5. The end cap 70 is cylindrically shaped with an outer surface circumference 72 similar in dimension to the third inner circumferential surface 58 of the contact housing 50 such that the end cap may fit within the third inner circumferential surface of the housing when assembled. Further, the end cap 70 includes a bore with an inner circumferential surface 74 of generally the same circumference as the first inner circumferential surface 54 of the housing 50. In one embodiment, the end cap 70 is constructed of stainless steel, titanium or any other electrically conductive material. In another embodiment, the end cap 70 is injection molded using a plastic material such as polysulfone or the like.

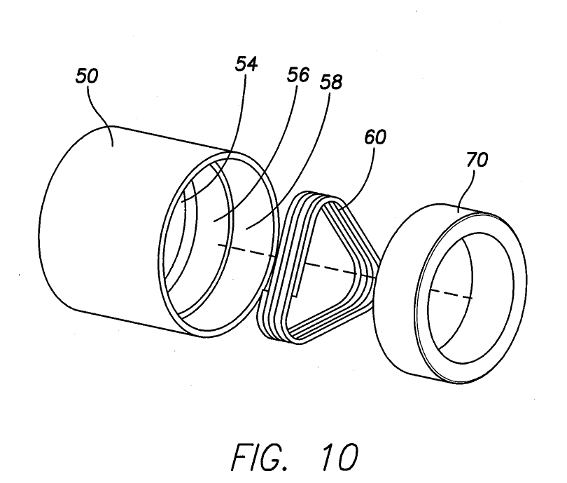

FIG. 10 is an exploded view of the assembly of the spring contact of the connector assembly, including the housing 50, the triangular metal spring 60 and the end cap 70. To assemble the spring contact, the metal spring 60 is inserted into a portion of the inner surface of the housing 50 defined by the second inner circumferential surface 56 of the housing and adjacent to the first inner circumferential surface 54 of the housing. When located within the housing, and as best seen in FIG. 11, the triangular shape of the metal spring 60 includes an inner spring boundary that protrudes into the bore 52 of the housing past the first inner circumferential surface. Specifically, the inner spring boundary that protrudes into the bore 52 is formed by the linear straight sides 84 of the spring 60 extending relative to the first inner circumferential surface much like a chord to a circle. At the same time, the curved corners or lobes 86 are recessed within the groove or slot 88 defined between the first inner circumferential surface 54 and the end cap 70 and defining the second inner circumferential surface 56, the lobes 86 making physical contact with the second inner circumferential surface 56.

During assembly, the end cap is inserted into the bore of the housing 50 defined by the third inner circumferential surface 58 of the housing and adjacent to the metal spring 60 and the second inner circumferential surface 56 of the housing. In one embodiment, the circumference of the outer surface of the end cap is similar to the third inner circumferential surface 58 such that the end cap 70 may be held in place within the housing 50 through a frictional force between the end cap and the inner surface of the housing. A cross-sectional view of the completed spring contact assembly spring contact is shown in FIG. 11.

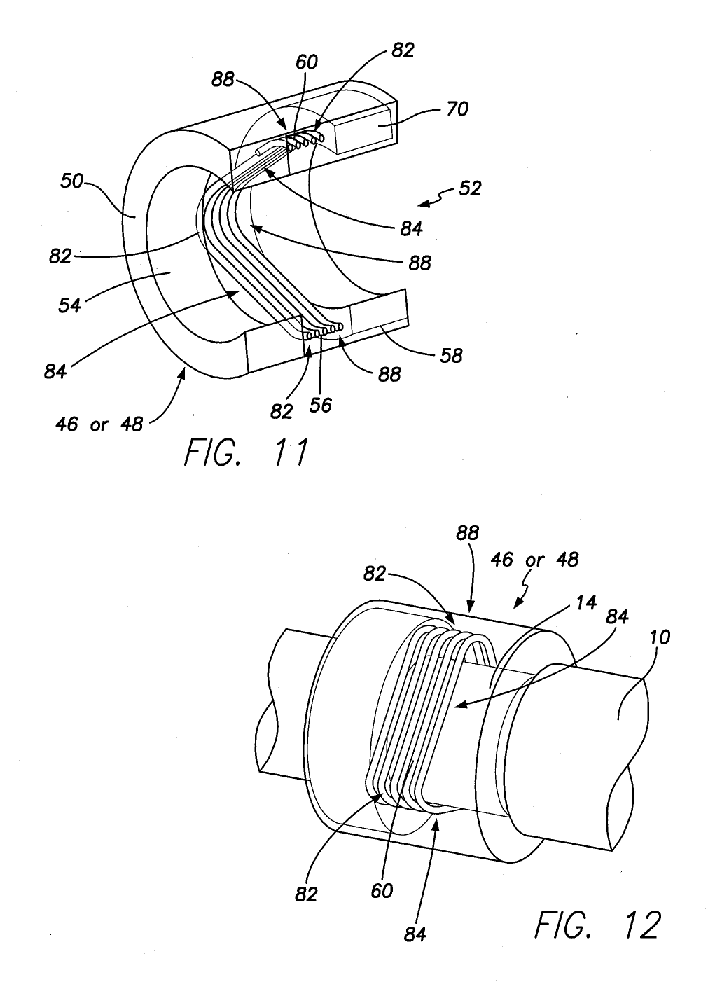

FIG. 11 is an isometric longitudinal cross-sectional view of the spring contact of the connector assembly along section line 11-11 of FIG. 5. Once assembled as described above, the spring contact 46 or 48 defines a cylindrical contact including a bore 52 for receiving at least part of the lead connector of FIG. 1. The trough or groove 88 is defined in the second inner circumferential surface 56 of the housing 50 and is oriented generally perpendicular to a center axis of the housing bore 52. The retaining sides of the groove 88 are generally defined by the first inner circumferential surface 56 of the housing and the end cap 70 and operate to retain the metal spring 60 within the groove 88 upon assembly. In this configuration, the three corners of the triangular shape are at least partially recessed in the groove 56 such that the metal spring remains in place within the spring contact during insertion and removal of the lead within the contact. Further, the triangular shape of the metal spring includes an inner spring boundary that protrudes into the bore 52 past the first inner circumferential surface 54 such that the inner spring boundary contacts the corresponding electrical terminal 12 or 14 of the lead connector end 10 when the end is fully received in receptacle 30 or 31 and corresponding bore 42 or 44. This can best be seen in FIG. 12, which shows an isometric view of connector of the pulse generator engaging a lead connector end on a proximal end of a lead 10. As shown in FIG. 12, the ring terminal 14 of the lead end portion 10 engages the metal spring 16 when engaged within the spring contact 46 or 48. Specifically, the outer circumferential surface of the electrical contact 14 (e.g., ring contact or pin contact) of the lead connector end 10 makes contact with the linear straight sides 84 of the spring 60, deflecting the linear straight sides 84 slightly outward and more fully driving the lobes 82 of the spring 60 into contact with the inner circumferential surface 56. Thus, as the spring contacts 46 or 48 are electrically coupled via feedthrough conductors and a feedthrough to the electrical components housed within the can 24 of the pulse generator, the electrical pulses are transmitted to the terminals of the lead connector end 10 of the lead for transmission through the lead.

As can be understood from FIG. 11, the triangular shape of the spring, with the tips 82 of the triangle being recessed in the groove 88 and the rest of the triangle (i.e., the straight sides 84) being exposed in the bore, results in the linear or straight sides 84 of the triangle extending into the bore. This also would be the case with square springs or other springs having straight segments. These alternate embodiments of the metal spring of the spring contact 46 or 48 may create an electrical connection with the lead in a similar manner. For example, a rectangular shaped metal spring may be retained within the groove 88 of the spring contact 46 or 48 at the corners of the rectangular shape. Also, similar to the triangular metal spring shape, the rectangular metal spring may include an inner surface of the spring that protrudes into the bore 52 of the spring contact to connect with the terminals of the inserted lead connector end 10 to transmit the generated electrical pulses from the pulse generator to the lead.

The spring contact design described above may provide several advantages over previous designs for electrically connecting an implantable medical lead to an implantable pulse generator. For example, a garter-type metal spring within the spring connector is generally more of a complex design and may require significant cost to manufacture compared to the helically wound non-circular spring described above. Further, due to the flexibility of some garter-type spring contacts, the garter-type springs may become dislodged from the spring contact housing during insertion and removal of the medial lead. In contrast, the spring contact described herein may be simpler and more cost-effective to manufacture. FIG. 13, for example, is an isometric view of an extended helically wound triangular metal spring 130 utilized for the triangular metal spring of the spring contact of FIG. 5. As described above, the extended triangular metal spring 130 is generally formed of a circular wire 61 helically wrapped or wound around a mandrel of the desired shape, such as a triangular or rectangular mandrel to create one or several loops of the wire that take the general shape of the mandrel. Alternatively, the spring 130 may be manufactured via CNC wire forming The extended metal spring 130 may then be cut to any length as desired, such as into metal springs of five or six loops. In this manner, the metal springs for several spring contacts may be quickly produced from a single wire wrapping or forming process. The extended triangular spring 130 of FIG. 13 has the same features as the triangular springs 60 made therefrom and shown in FIG. 8. In addition, because the triangular spring design is generally less laterally flexible than other spring designs, such as, for example, a garter-type spring, the potential for dislodgment of the metal spring from the spring contact is reduced during insertion and removal of the medial lead from the spring contact. In addition, the metal spring design described herein provides several contact points between the spring and the terminals 12 and 14 of the electrical lead 10.

The contact between a garter-type spring and an electrical contact of a lead connector end and the contact between the herein-disclosed non-circular spring and an electrical contact of a lead connector end are different, this difference at least in part making the herein-disclosed non-circular spring less likely to dislodge from the groove when the lead connector end is inserted into, or removed from, the lead receptacle hole of the header. For example, the garter-type spring is a wire helically wound in a ring such that a longitudinal axis of the helix extends in a ring about the lead connector end ring contact, the longitudinal axis of the helix of the garter-type spring residing in a plane that is generally perpendicular to a longitudinal axis of a lead connector end extending through the garter-type ring. As a result of its configuration and orientation, the garter-type spring can be said to have a rolling type contact with the lead connector end as the lead connector end is inserted into, or removed from within, the ring formed by the garter-type spring. This rolling contact, coupled with its high flexibility, may contribute to the garter-type spring's higher likelihood of dislodgement as compared to the herein-disclosed, non-circular spring.

As can be understood from FIGS. 4, 11 and 12, the herein-disclosed, non-circular spring is oriented in the spring connector and lead receptacle hole of the header such that the longitudinal axis of the helical winding of the non-circular spring is generally coaxial with the longitudinal axes of the spring connector and lead receptacle hole of the header. Thus, the lead connector end is received through the center of the helical windings of the non-circular spring as opposed to outside the helical windings as is the case with a garter-type spring.

FIG. 14 is an isometric view of a second embodiment of a triangular metal spring of the connector of the pulse generator. In this embodiment, the metal spring 140 includes one or more single metal loops 141 of a particular shape, such as a triangular shape. Thus, this embodiment is similar to the metal spring embodiment described above. However, rather than forming the metal spring from a single circular wire wound around a mandrel or manufactured via CNC wire forming, this embodiment may be formed from one or more individual single loop metal springs 140 stacked or otherwise oriented into a stack within the housing. Upon assembly, the one or more metal loops 140 are stacked within the housing 50 in a similar manner as described above to form the metal spring within the spring contact. Other aspects of the spring contact described above are similar between the two embodiments.

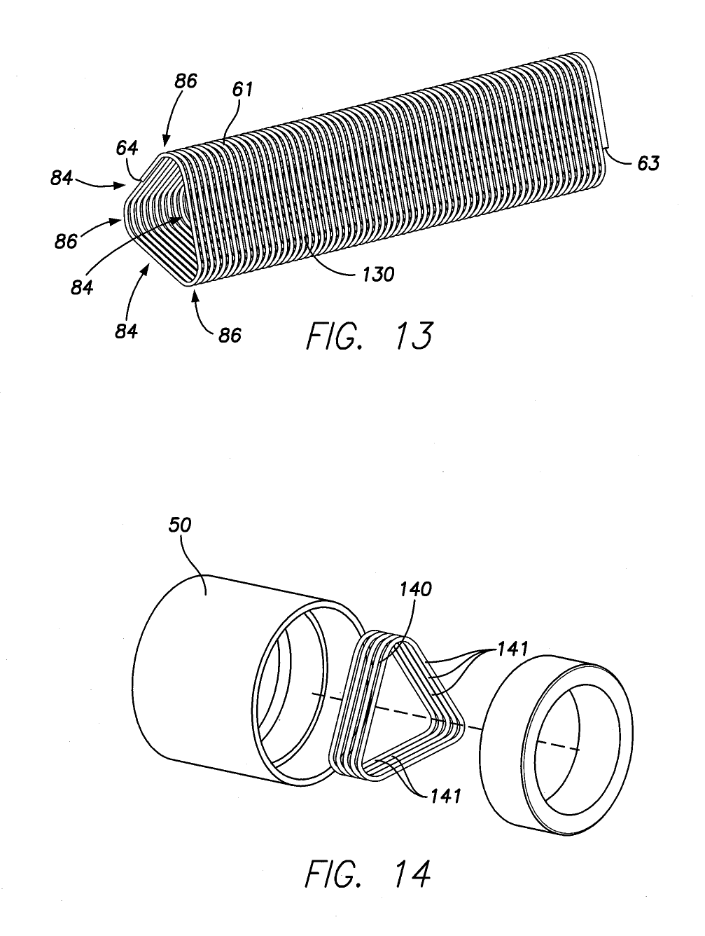

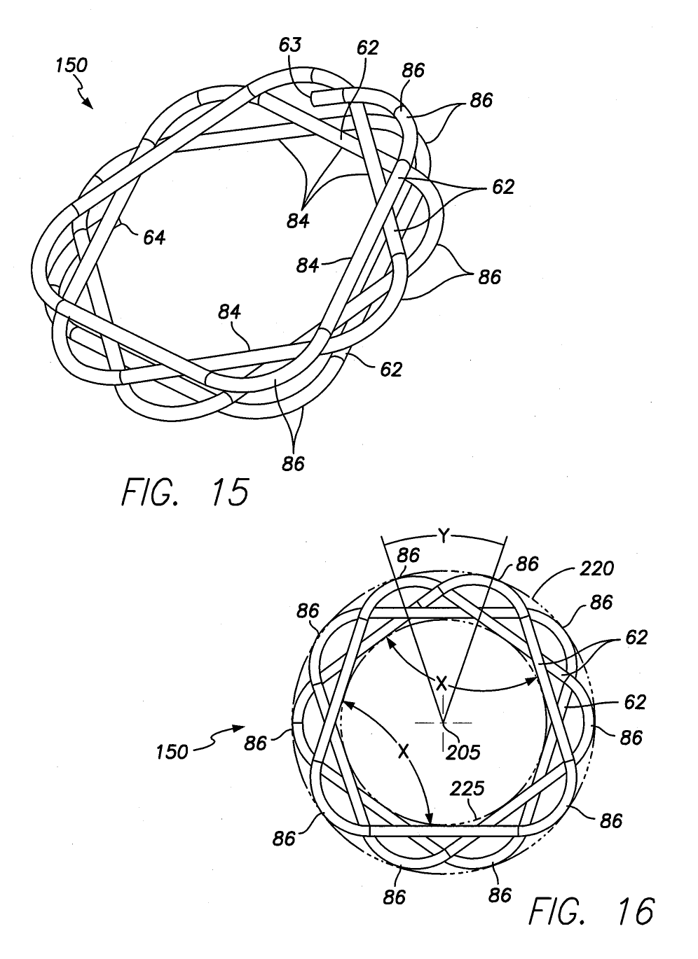

FIGS. 15-17 are, respectively, an isometric view, an axial end elevation, and a side elevation of a third embodiment of the metal spring 150 of the spring contact, wherein the spring has a non-circular transverse cross section and the coils of the spring are radially offset from each other. As can be understood from FIG. 4 and as is the case with the above-described springs, when the spring 150 of FIGS. 15-17 is part of a conductive spring contact 46, 48, the longitudinal axis 200 of the bore 42, 44 of the lead connector end receiving receptacle 30, 31 is at least generally coaxial with the longitudinal axis 205 of the spring 150. As a result and as discussed above, the lead connector end is received through the center of the helical windings of the non-circular spring as opposed to outside the helical windings as is the case with a garter-type spring

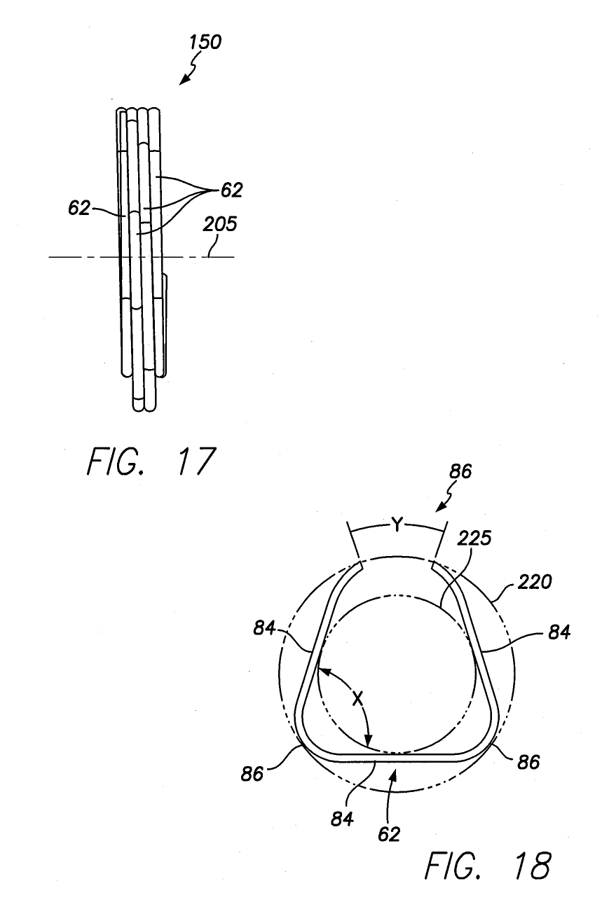

As indicated in FIGS. 15-17, the spring 150 includes multiple coils 62 helically extending about the longitudinal axis 205 of the spring 150 as a continuous wire formed into the spring via CNC wire forming between the ends 63, 64 of the wire. As best understood from FIG. 16, the coils 62 have a non-circular shape when viewed along the longitudinal axis of the spring, and the coils are radially offset or staggered relative to each other about the longitudinal axis 205 such that the corners 86 of one coil 62 are radially offset from the corners of the immediately adjacent coils.

As can be understood from FIG. 18, which is an axial end elevation of a single loop or coil 62 of the spring 150 of FIGS. 15-17, the non-circular shape illustrated includes a generally triangular shape. In one embodiment, the generally triangular shape is generally equal lateral in nature such that the sides 84 are generally equal and the angles X of the corners are generally equal.

In one embodiment, the angles X of the corners 86 of each generally triangular shaped coil 62 are equal to each other and each greater than 60 degrees such that the total angular displacement of a coil 62 is greater than the 180 degrees of a true triangle, resulting in the next adjacent coil 62 being radially offset about the longitudinal axis 205 of the spring 150 by the amount Y of the total angular displacement that exceeds the 180 degrees of a true triangle. Thus, as an example, a generally triangular coil 62 with three corners each having an angle X of 72 degrees results in a total angular displacement of the coil of 216 degrees, which exceeds the 180 degrees of a true triangle by an amount Y of 36 degrees. As a result, the next adjacent generally triangular shaped coil 62 has a radial offset Y of 36 degrees about the longitudinal axis 205 from the preceding generally triangular shaped coil.

Thus, as can be understood from FIG. 16, the center of each corner 86 of each coil 62 is radially offset from the two immediately radially adjacent corners 86 of the adjacent coils 62 by a radial offset Y of 36 degrees. As a result, such a spring 150 has ten corners 86 evenly radially distributed about the outer circumference 220 of the spring 150 when the spring is viewed coaxially along the longitudinal axis 205 of the spring, as shown in FIG. 16.

In one embodiment, the outer circumference 220 of the each coil 62 of the spring 150 has a diameter of between approximately 0.07 inch and approximately 0.16 inch, with one embodiment having a diameter of approximately 0.147 inch. The outer circumference 220 is a circle that tangentially intersects the extreme points of the corners 86. The inner circumference 225 of the each coil 62 of the spring 150 has a diameter of between approximately 0.05 inch and approximately 0.12 inch, with one embodiment having a diameter of approximately 0.1 inch. The inner circumference 225 is a circle that tangentially intersects the center points of each side 84.

In one embodiment, each corner 86 may be generally arcuate or curved and have a radius of between approximately 0.01 inch and approximately 0.04 inch, with one embodiment having a radius of approximately 0.24 inch. In one embodiment, the pitch of the helical winding of the spring may be between approximately 0.004 inch and approximately 0.008 inch, with one embodiment having a pitch of approximately 0.005. In one embodiment, the spring 150 may have between approximately 3 and approximately 10 coils 62, with one embodiment having approximately four coils 62. In one embodiment, the spring 150 may have a length along the longitudinal axis 205 of between approximately 0.015 inch and approximately 0.05 inch, with one embodiment having a length of approximately 0.023 inch. In one embodiment, the spring 150 may formed of a wire with a diameter of between approximately 0.004 inch and approximately 0.006 inch, with one embodiment having a diameter of approximately 0.005 inch. In one embodiment, the wire may be MP35N, stainless steel, etc.

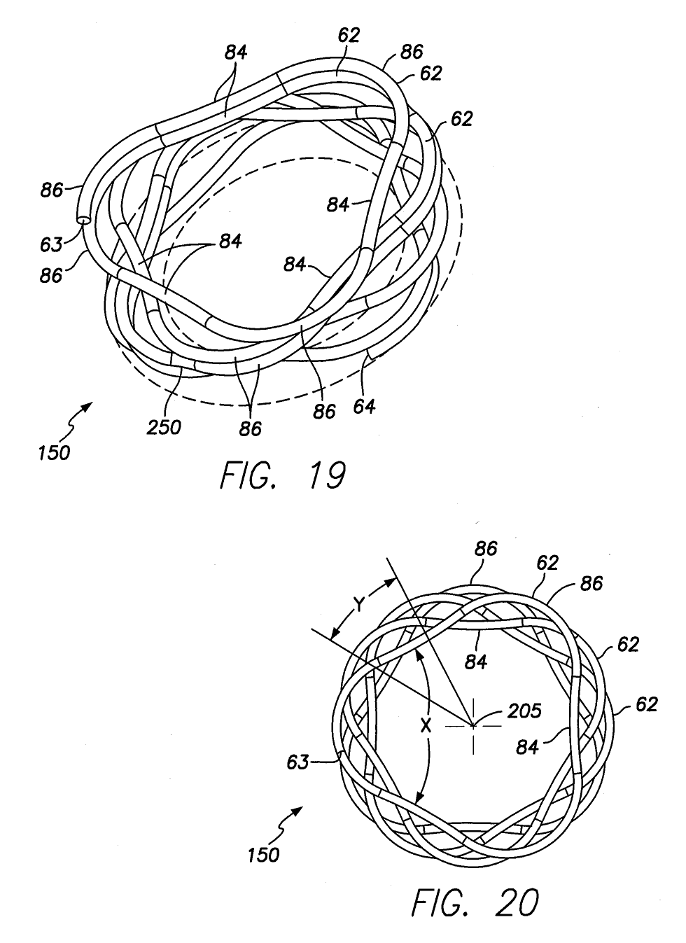

FIGS. 19-21 are, respectively, an isometric view, an axial end elevation, and a side elevation of a fourth embodiment of the metal spring 150 of the spring contact, wherein the spring has a non-circular transverse cross section and the coils of the spring are radially offset from each other. As can be understood from FIG. 4 and as is the case with the above-described springs, when the spring 150 of FIGS. 19-21 is part of a conductive spring contact 46, 48, the longitudinal axis 200 of the bore 42, 44 of the lead connector end receiving receptacle 30, 31 is at least generally coaxial with the longitudinal axis 205 of the spring 150. As a result and as discussed above, the lead connector end is received through the center of the helical windings of the non-circular spring as opposed to outside the helical windings as is the case with a garter-type spring.

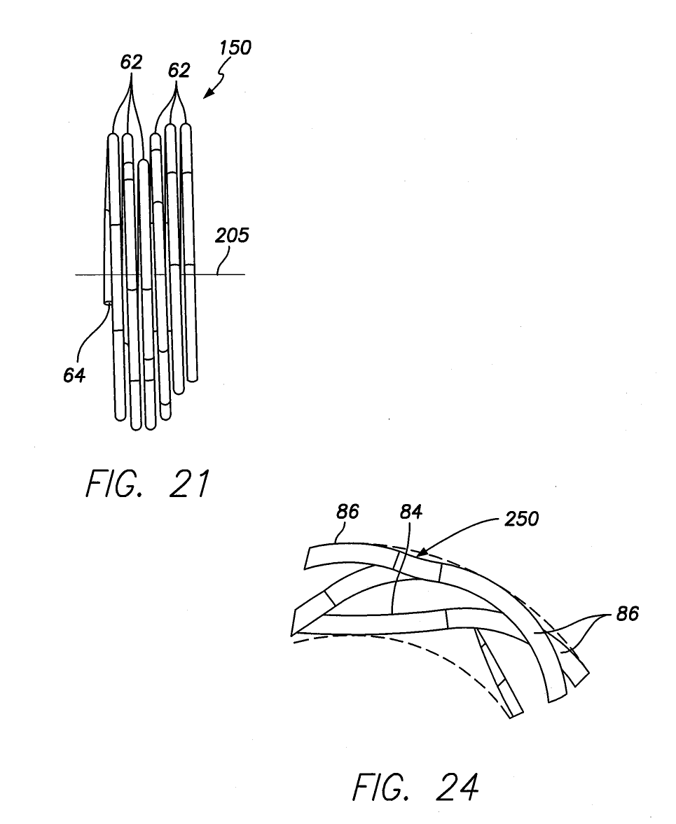

As indicated in FIGS. 19-21, the spring 150 includes multiple coils 62 helically extending about the longitudinal axis 205 of the spring 150 as a continuous wire formed into the spring via CNC wire forming between the ends 63, 64 of the wire. As best understood from FIG. 20, the coils 62 have a non-circular shape when viewed along the longitudinal axis of the spring, and the coils are radially offset or staggered relative to each other about the longitudinal axis 205 such that the corners 86 of one coil 62 are radially offset from the corners of the immediately adjacent coils.

As can be understood from FIG. 20, the non-circular shape of the individual coils 62 forming the spring 150 includes a generally triangular shape. In one embodiment, the generally triangular shape is generally equal lateral in nature such that the sides 84 are generally equal and the angles X of the corners are generally equal.

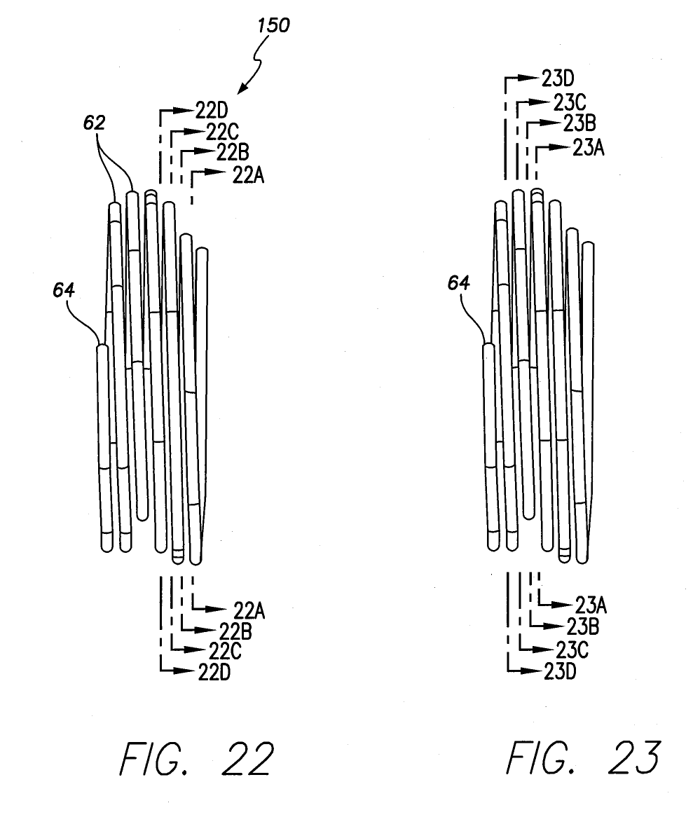

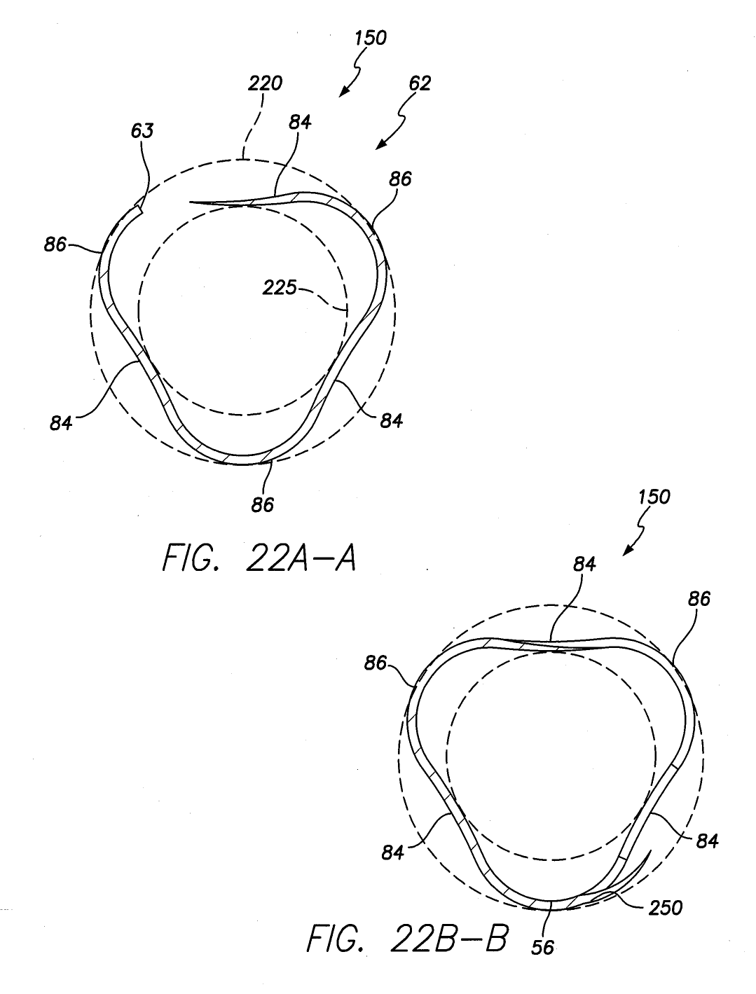

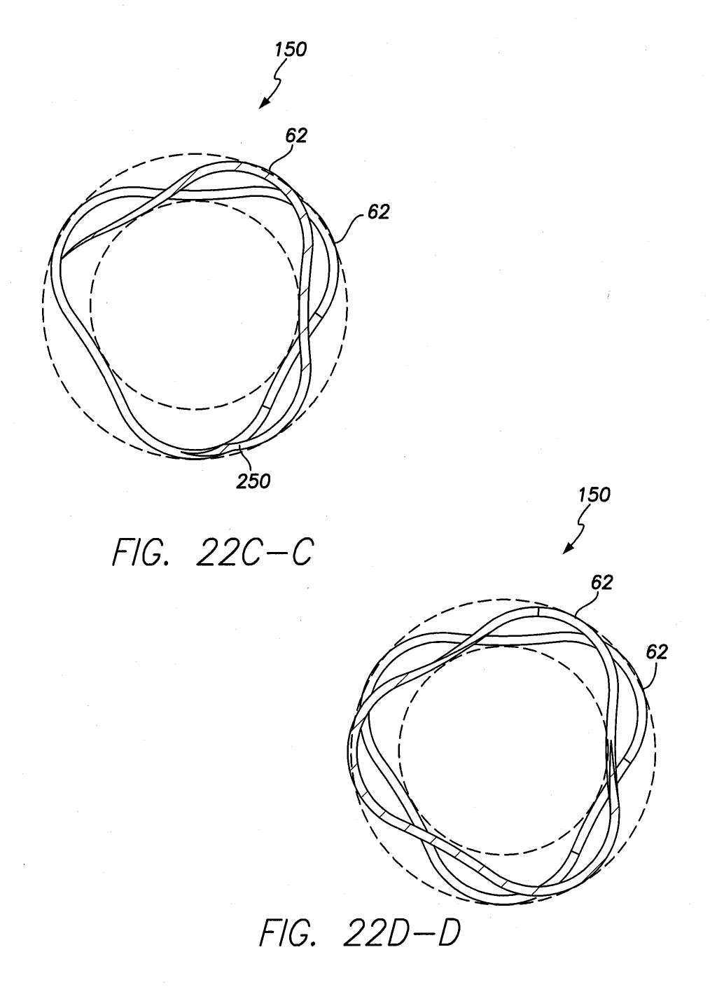

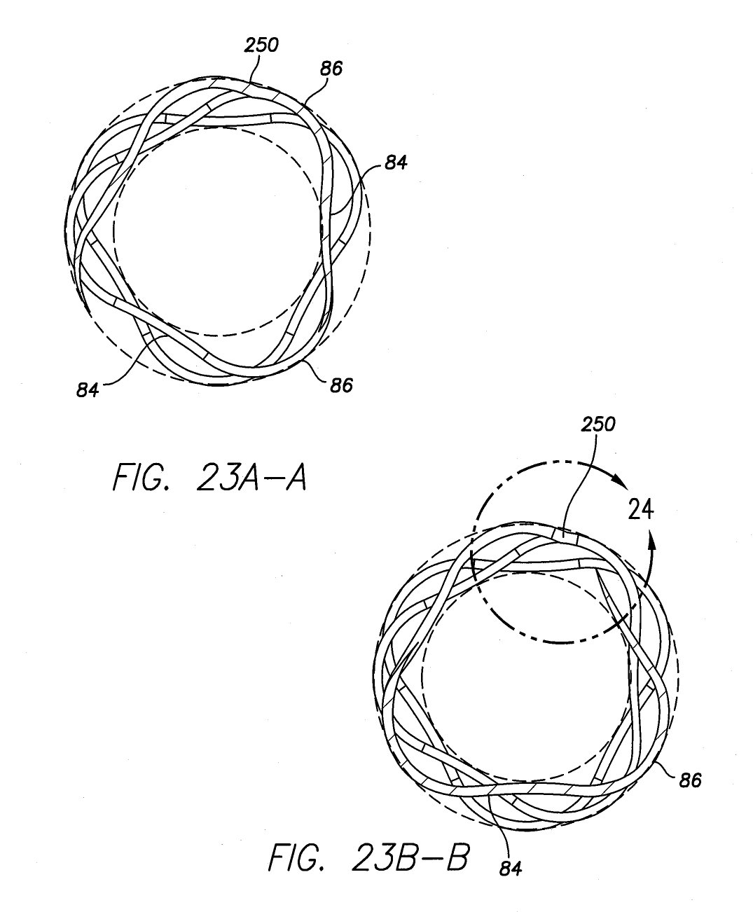

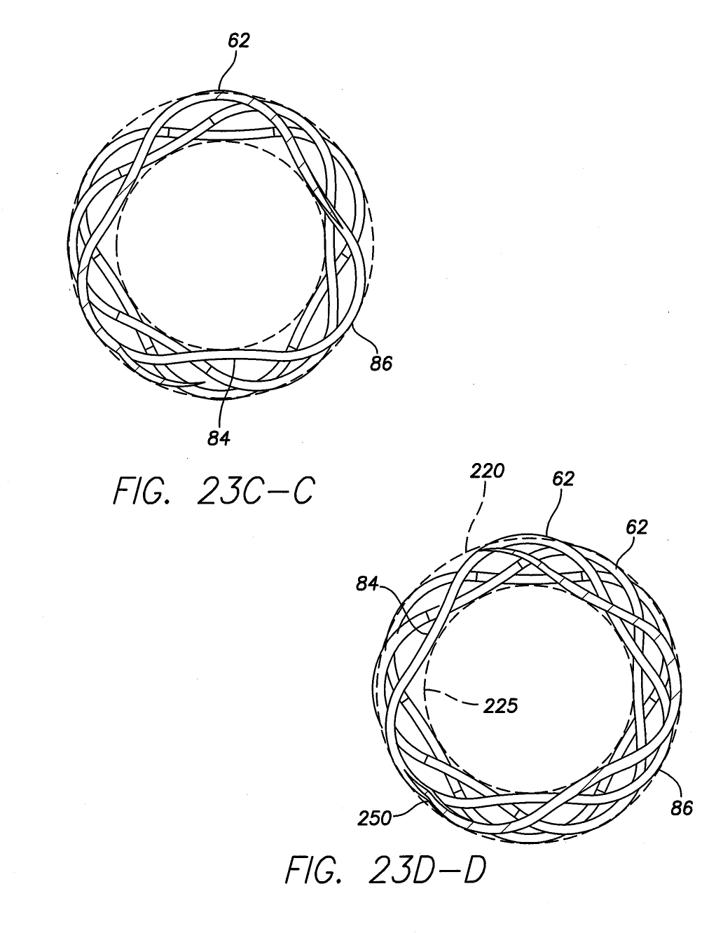

As can be understood from FIG. 20, in one embodiment, the angles X of the corners 86 of each generally triangular shaped coil 62 are equal to each other and each approximately 60 degrees. As illustrated in FIG. 19, each individual coil 62 of the spring 150 includes corners 86 and sides 84 extending between the corners. In one embodiment, a coil angular arrangement for an individual coil 62 extends along the full coil plus a full side 84 and a corner 86 of the next immediately adjacent coil 62 before the coil angular arrangement changes via a transitional segment 250. Thus, as can be understood from the side elevation view of the spring 150 in FIG. 22 and its transverse cross sections FIGS. 22A-A, 22B-B, 22C-C and 22D-D respectively taken along section lines A-A, B-B, C-C and D-D of FIG. 22 and, as can be further understood from the side elevation view of the spring 150 in FIG. 23 and its transverse cross sections FIGS. 23A-A, 23B-B, 23C-C and 23D-D respectively taken along section lines A-A, B-B, C-C and D-D of FIG. 23, a first generally triangular shaped coil 62 will helically extend about the longitudinal axis 205 of the spring 150 along the three sides 84 and three corners 86 of the first coil 62 and into the first side 84 and corner 86 of the next adjacent (e.g., second) generally triangular shaped coil 62 before there will be a transition segment or zone 250 in the helically extending wire, wherein the angular arrangement changes, thereby resulting in the next (e.g., second) coil 62 being radially offset from the first coil 62 about the longitudinal axis 205 of the spring 150.

More specifically, as indicated in FIG. 22A-A, a first generally triangular coil 62 helically extends about the longitudinal axis 205 of the spring 150 from a first free end 63, the first coil 62 including three sides 84 and three corners 86. As can be understood from a comparison of FIGS. 22A-A and 22B-B, the angular arrangement begun with the first coil in FIG. 22A-A continues for a first side 84 and first corner 86 of the second coil 62 in FIG. 22B-B before encountering the first transition zone 250, wherein a new angular arrangement begins. The difference between the angular arrangements of the first two coils 62 can be seen in FIG. 22C-C. As can be understood from FIG. 22D-D and FIG. 23A-A, the second angular arrangement continues for three full sides 84 and corners 86 of the second coil 62 and into the first side 84 and corner 86 of the next adjacent (e.g., third) coil 62 before another transition 250 and change in the angular arrangement between coils will be encountered. As can be understood from FIG. 23B-B, FIG. 23C-C and FIG. 23D-D, the third angular arrangement continues for three full sides 84 and corners 86 of the third coil 62 and into the first side 84 and corner 86 of the next adjacent (e.g., fourth) coil 62 before another transition 250 and change in the angular arrangement between coils will be encountered.

In one embodiment, as can be understood from FIG. 24, which is an enlarged view of region A in FIG. 23B-B, transition 250 may be generally located near the middle or center of a corner 86, the transition 250 being a curve or bend in the wire that is opposite the curve or bend of the rest of the corner 86 in which the transition 250 is located.

In one embodiment, the transition 250 may have a radius of between approximately 0.03 inch and approximately 0.06 inch, with one embodiment having a radius of approximately 0.048 inch. Such a radius may provide a change in angular arrangement between coils of between approximately 25° and approximately 35°, with said 0.048 inch radius providing a change in angular arrangement between coils of approximately 30 degrees.

Thus, as can be understood from FIG. 20, the center of each corner 86 of each coil 62 is radially offset from the two immediately radially adjacent corners 86 of the adjacent coils 62 by a radial offset Y of 30 degrees due to the presence of the transition 250, which may have, for example, a radius of approximately 0.048 inch. As a result, such a spring 150 has twelve corners 86 evenly radially distributed about the outer circumference 220 of the spring 150 when the spring is viewed coaxially along the longitudinal axis 205 of the spring, as shown in FIG. 20.

In one embodiment as can be understood from 22A-A through 23D-D, the outer circumference 220 of the each coil 62 of the spring 150 has a diameter of between approximately 0.07 inch and approximately 0.16 inch, with one embodiment having a diameter of approximately 0.147 inch. The outer circumference 220 is a circle that tangentially intersects the extreme points of the corners 86. The inner circumference 225 of the each coil 62 of the spring 150 has a diameter of between approximately 0.05 inch and approximately 0.12 inch, with one embodiment having a diameter of approximately 0.1 inch. The inner circumference 225 is a circle that tangentially intersects the center points of each side 84.

In one embodiment as can be understood from 22A-A through 23D-D, each corner 86 may be generally arcuate or curved and have a radius of between approximately 0.01 inch and approximately 0.04 inch, with one embodiment having a radius of approximately 0.24 inch. In one embodiment, the pitch of the helical winding of the spring may be between approximately 0.004 and approximately 0.008, with one embodiment having a pitch of approximately 0.005. In one embodiment, the spring 150 may have between approximately 3 and approximately 10 coils 62, with one embodiment having approximately four coils 62. In one embodiment, the spring 150 may have a length along the longitudinal axis 205 of between approximately 0.015 inch and approximately 0.05 inch, with one embodiment having a length of approximately 0.023 inch. In one embodiment, the spring 150 may formed of a wire with a diameter of between approximately 0.004 inch and approximately 0.006 inch, with one embodiment having a diameter of approximately 0.005 inch. In one embodiment, the wire may be MP35N, stainless steel, etc.

While the preceding embodiments of the springs 150 have been discussed in the context of having coils 62 that are generally triangular in shape, in other embodiments, the non-circular shaped coils 62 of the springs 150 may include ovals, rectangles, pentagons, etc. For example, the non-circular shape could simply be a coil with a portion thereof defined by two sides 84 joined together by a corner 86. The sides 84 may be generally straight as indicated in FIGS. 15, 16 and 18, or the sides 84 may be curved slightly radially inward, as illustrated in FIGS. 19, 20, and 22A-A through 23D-D.

Regardless of what non-circular shape the coils 62 of a spring 150 may have, the spring, when viewed along the longitudinal axis 205 of the spring 150, will include a plurality of corners 86, the corners 86 being defined on multiple coils 62 of the spring. For example, a spring 150 with triangular shaped coils 62 may have a plurality of corners, when viewed along the longitudinal axis 205 that is between approximately 9 and approximately 30 corners. Ten and twelve corner embodiments of the springs 150 are depicted, respectively, in FIGS. 16 and 20.

As can be understood from FIGS. 1, 3 and 4, when the lead connector end 10 is inserted into the bore 42 of a lead end receiving receptacle 30, the pin contact 12 and ring contact 14 of the lead connector end 10 extend into their respective spring contacts 46 and 48. As a result, the longitudinal centers of the lead connector end 10, the bore 42 and the springs 150 end up being generally coaxially aligned with each other.

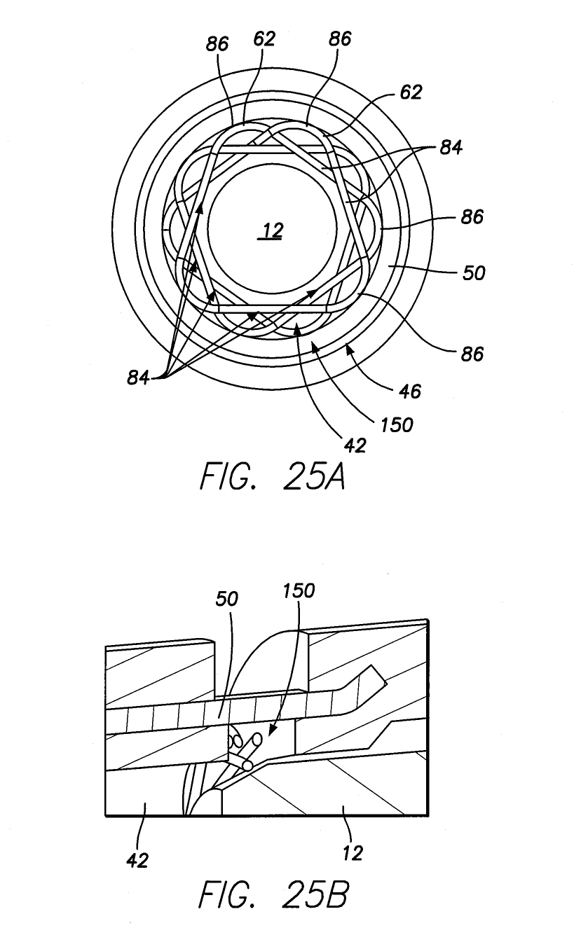

As can be understood from FIGS. 25A and 25B, which are, respectively, an axial view and a partial longitudinal cross section of the proximal end (i.e., pin contact 12) of the lead connector end 10 entering the spring contact 46 and just beginning to make contact with the spring 150, the generally straight sides 84 of the coils 62 of the spring 150 begin to make contact at their center points with the leading beveled edge of the pin contact 12. The inner circumference diameter 225 (see, for example, FIG. 16) is can be seen in FIGS. 25A and 25B to be slightly less than the outer diameter of the pin contact 12. As a result, the pin contact 12 will need to deflect the sides 84 of the coils 62 of the spring 150 if the pin contact 12 is further inserted into the bore 46.

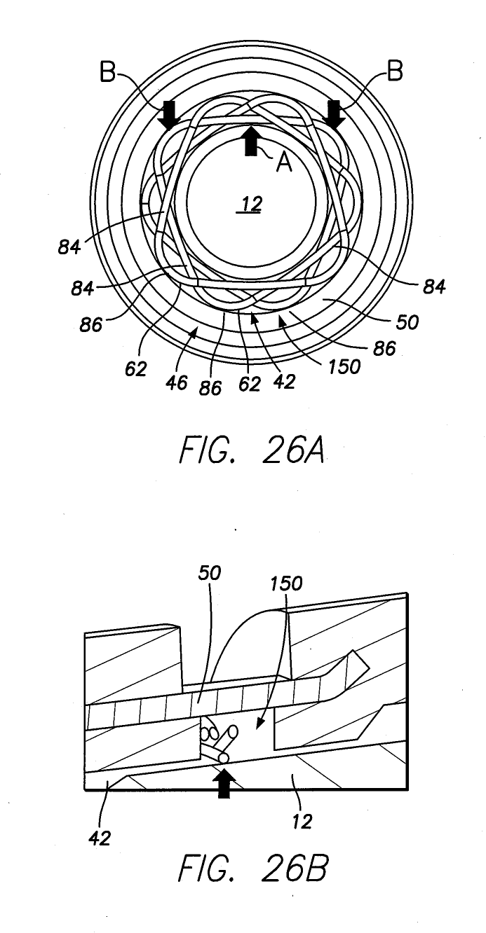

As can be understood from FIGS. 26A and 26B, which are, respectively, the same views as FIGS. 25A and 25B, except with the pin contact 12 of the lead connector end 10 received within the spring 150 of the spring contact 46, the generally straight sides 84 of the coils 62 of the spring 150 have been deflected outwardly via contact with the outer circumferential surface of the pin contact 46. As indicated by arrow A, the interference between the outer circumferential surface of the pin contact 12 and the sides 84 of the coils 62 of the spring 150 causes the sides 84 to slightly bow radially outwardly. This strong interference engagement between the sides 84 and the outer circumferential surface of the pin 12 provides a large number of strong electrical contacts between the spring 150 and the pin contact 12. As indicated by arrows B, the bowing or deflecting force brought to bear against the sides 84 by the pin contact 12 tends to drive the corners 86 of the coils 62 of the spring 150 more fully against the conductive ring 50 of the spring contact 46, thereby providing a large number of strong electrical contacts between the spring 150 and the conductive ring 50 of the spring contact 46.

In one embodiment, the spring 150 of the spring contact 46 is formed via a continuous process. For example, a continuous wire is fed from a wire spool into a CNC wire forming machine. The CNC wire forming machine helically winds the continuously extending wire into a helix having a continuous wire. In doing so, the CNC wire forming machine causes the continuously extending wire to be formed into multiple coils of the helix, the coils being formed to have a non-circular shape when viewed along a longitudinal axis of the helix. The resulting helix may have a length similar to that depicted in FIG. 13 or even much greater, wherein multiple springs can be generated from the helix. The helix may be stored on racks or spools until an individual spring needs to be cut from the helix. The resulting helix may have an oval, triangular, square, pentagonal, hexagonal, etc. axial appearance. Alternatively, the resulting helix may have an axial appearance as depicted in FIG. 16 or 20.

To manufacture garter-springs known in the art, helical windings must be cut to a desired length and then welded end-to-end to form the garter-springs. Estimating the correct length to be cut for a garter-spring is more difficult than the estimating needed for the springs disclosed herein. Also, unlike garter-springs, the springs disclosed herein require no welding. For at least these reasons, the springs disclosed herein and associated continuous wire forming process used to make such springs are advantageous over garter-springs and their methods of manufacture known in the art.

Once the helix is formed as described, a cutting operation can be applied to the helix to cut a helical spring 150 of a desired length from the helix. The helical spring is then positioned in an electrically conductive ring 50 of the spring contact 46 of the lead end connector receiving receptacle of the pulse generator so a longitudinal axis of the spring is generally coaxially aligned with the longitudinal axis of the ring and bore of the lead end connector receiving receptacle.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. An implantable medical pulse generator for administering electrotherapy via an implantable medical lead having a lead connector end on a proximal end of the lead, the lead connector having a terminal, the pulse generator comprising:
a can; and
a header coupled to the can, the header comprising:
a lead connector end receiving receptacle; and
a conductive spring contact associated with the lead connector end receiving receptacle and configured to be in electrical contact with the terminal of the first lead connector end when the lead connector end is inserted into the lead connector end receiving receptacle;
wherein the conductive spring contact comprises:
a cylindrically shaped housing including a bore through the body of the housing; and
a triangularly shaped metal spring located at least partially within the bore of the housing and oriented such that at least an inner portion of the spring protrudes into the bore of the housing.

2. The pulse generator of claim 1 wherein the bore of the housing includes a stepped bore.

3. The pulse generator of claim 2 wherein the conductive spring contact further comprises:
an end cap substantially adjacent the metal spring within the stepped bore of the housing.

4. The pulse generator of claim 3 wherein the stepped bore of the housing defines a first inner circumferential surface, a second inner circumferential surface with a larger circumference than the first inner circumferential surface and a third inner circumferential surface with a larger circumference than the second inner circumferential surface.

5. The pulse generator of claim 4 wherein the metal spring is substantially located within the area defined by the second inner circumferential surface.

6. The pulse generator of claim 4 wherein the end cap is substantially located within the area defined by the third inner circumferential surface.

7. The pulse generator of claim 4 wherein the first inner circumferential surface and the end cap define a groove within the stepped bore of the housing, and wherein the corners of the triangularly shaped metal spring are at least partially recessed within the groove.

8. The pulse generator of claim 1 wherein the triangularly shaped metal spring comprises a circular wire formed into a plurality of loops, each loop of a generally triangular shape.

9. The pulse generator of claim 1 wherein the triangularly shaped metal spring comprises a plurality of triangularly shaped wire loops located substantially adjacent each other within the housing.

10. The pulse generator of claim 2 wherein the inner portion of the spring protruding into the stepped bore of the housing includes a straight segment of the spring.

11. An implantable medical pulse generator comprising:
a can; and
a header coupled to the can, the header comprising:
a lead connector end receiving receptacle; and
a spring contact associated with the lead connector end receiving receptacle;
wherein the spring contact comprises:
a cylindrically shaped housing including a bore through the body of the housing; and
a non-circular shaped metal spring located at least partially within the bore of the housing and oriented such that at least an inner portion of the spring protrudes into the bore of the housing.

12. The pulse generator of claim 11 wherein the non-circular shaped metal spring comprises a circular wire formed into a plurality of triangularly shaped loops.

13. The pulse generator of claim 11 wherein the non-circular shaped metal spring comprises a circular wire formed into a plurality of rectangularly shaped loops.

14. The pulse generator of claim 11 wherein the spring contact includes a groove within the bore of the housing, and wherein the corners of the non-circular shaped metal spring are at least partially recessed within the groove.

15. The pulse generator of claim 11 wherein the spring contact is configured to be in electrical contact with at least one terminal of a lead connector end when inserted into the lead connector end receiving receptacle.

16. The pulse generator of claim 13 wherein the inner portion of the spring protruding into the bore of the housing includes a straight segment of the spring.

17. An apparatus for administering electrotherapy via an implantable medical lead having a lead connector end on a proximal end of the lead, the lead connector end including a terminal, the apparatus comprising:
a can configured to generate an electrical pulse; and
a connector assembly configured to transmit the generated electrical pulse to the lead connector end of the implantable medical lead through a conductive spring contact in electrical communication with the terminal of the lead connector;
wherein the conductive spring contact comprises:
a cylindrically shaped housing including a bore through the body of the housing of at least a first circumference;
a groove located within the bore of the housing, the groove having a circumference larger than the first circumference; and
a triangularly shaped metal spring located at least partially within the groove and oriented such that the corners of the triangularly shaped metal spring are at least partially recessed within the groove.

18. The apparatus of claim 17 wherein the metal spring comprises an inner surface that at least partially protrudes into the bore of the housing to form an electrical communication with the terminal of the lead connector end when the lead connector end is inserted into the connector assembly.

19. The apparatus of claim 17 wherein the triangularly shaped metal spring comprises a circular wire formed into a plurality of loops, each loop of a generally triangular shape.

20. The apparatus of claim 17 wherein the triangularly shaped metal spring comprises a plurality of triangularly shaped wire loops located substantially adjacent each other within the housing.
